(19) United States
(12) Patent Application Publication
(10) Pub. No.: US 2004/0175421 A1
(43) Pub. Date: Sep. 9, 2004

(54) Solid oral dosage form of metformin and glyburide and the method of preparation thereof

(76) Inventors: Suresh Kumar Gidwani, Mumbai (IN); Purushottam Sharshikant Singnurkar, Maharashtra (IN)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/794,993

(22) Filed: Mar. 5, 2004

Related U.S. Application Data
(60) Provisional application No. 60/451,989, filed on Mar. 5, 2003.

(51) Int. Cl. A61K 31/175; A61K 31/155; A61K 31/535

(52) U.S. Cl. 424/465; 514/592; 514/635

(57) ABSTRACT

The present invention relates to a simple and easy method for the preparation of solid oral dosages of the combination of metformin and glyburide. Specifically, glyburide is homogenized in an organic solution whereby about 8 to 15% w/w of glyburide goes into solution and form a slurry. The resulted slurry is then sprayed onto pharmaceutical excipients or metformin granules, and the solid oral dosage of metformin and glyburide is formed. The present invention further relates to the solid oral dosages prepared by the disclosed process, and the method of treating non-insulin dependent diabetes by administering to a patient in need thereof a solid oral dosage prepared by the current invention.

### CROSS REFERENCE TO RELATED APPLICATIONS

This application claims priority from provisional application U.S. Serial No. 60/451,989, filed on Mar. 5, 2003.

### BACKGROUND OF INVENTION

Glyburide and metformin are two types of oral antidiabetic medicines, belonging to the groups of sulphonylureas and biguanidines, respectively. Glyburide, also known as gliblenclamide, has the chemical structure of 1-[[p-[2-(5-chloro-o anisamido)ethyl]phenyl]sulfonyl]-3-cyclohexylurea. Glyburide lowers blood glucose acutely by stimulating the release of insulin from the pancreas, an effect dependent upon functioning beta cells in the pancreatic islets. Metformin, often in the form of hydrochloride salt, is chemically known as N,N-dimethylimidodicarbonimidic diamide hydrochloride. Metformin decreases hepatic glucose production, decreases intestinal absorption of glucose, and improves insulin sensitivity by increasing peripheral glucose uptake and utilization.

Due to the complementary modes of action, glyburide and metformin are often administered at the same time to patients with diabetes. The combination therapy is particularly advantageous to patients who have been treated by glyburide or metformin separately for a period of time and the treatment has become ineffective.

It is also desirable to combine glyburide and metformin into one single dosage form such as a tablet or a gelatin capsule. However, although metformin is highly soluble in water, glyburide is a weak acid and sparingly dissolves in water. Further, when combining glyburide and metformin into a single dosage form, the dissolution of glyburide is further reduced due to the presence of metformin in close vicinity to glyburide. Therefore, it is desirable to achieve a combination dosage of metformin and glyburide that has improved glyburide dissolvability. Preferably, the combination dosage would exhibit an in-vitro dissolvability and/or in-vivo bioavailability that are comparable to the glyburide dissolvability and/or bioavailability obtained by separately administering metformin and glyburide of the same dosage. In the alternative, it is desirable to have the combination dosage of metformin and glyburide to exhibit an in-vitro dissolvability and/or in-vivo bioavailability that are comparable to those obtained by administering the same dosage of the commercially available tablets containing metformin and glyburide, i.e. the Glucovance™ tablets approved by the United States Food and Drug Agency (FDA).

WO 97/17975 discloses that, in a combination of glyburide with metformin hydrochloride, a ratio of 1/100 by weight is required to obtain an optimum therapeutic effect in treating type II diabetes. It however does not teach how to formulate a combination product of metformin and glyburide so as to assure an appropriate in-vitro dissolvability and in-vivo bioavailability.

U.S. Pat. No. 5,258,185 describes a highly active, rapidly absorbable formulation of glyburide, consisting essentially of a solution of 1 part of glyburide, 2 to 30 parts of an aliphatic alcohol or mixtures thereof, 3 to 70 parts of at least one sugar alcohol, wherein said sugar alcohol is dissolved in 10 to 100 parts of water, and 0.5 to 2 moles of an alkali reacting substances e.g., alkali hydroxides, ammonia. By forming sodium salts with the alkali reacting substances, glyburide becomes molecularly dispersed, enabling a rapid absorption of glyburide from gastrointestinal tract. However, the formation of sodium salts is pharmacologically unacceptable because the sodium slats may show different pharmacokinetic parameters as comparing with glyburide itself. Additionally, the combination product disclosed in WO 01/51463 A1 patent fails to achieve the required bioavailability as it causes rapid onset of high drug concentration (the “spike”) in blood plasma.

U.S. Pat. No. 6,303,146 B1 and WO 01/51463 A1 disclose the effect of particle size and particle size distribution of glyburide on the in-vitro dissolvability and in-vivo bioavailability of glyburide. In U.S. Pat. No. 6,303,146 B1, it has been shown that, in order to achieve an appropriate in-vivo bioavailability of glyburide, the particle size of glyburide is such that at most 10% of the particles are less than 2 μm and at most 10% of the particles are greater than 60 μm. Similarly, WO 01/51463 A1 shows that in order to achieve the same result, the glyburide particles need to be in such a form that 25% of the particles are under size value between 3 and 11 μm, 50% of the particles are under size value between 6 and 23 μm, and 75% of the particles are under size value between 15 and 46 μm, with the powder surface area values in the range of about 1.7 to 2.2 square meter per gram. The currently marketed product “Glucovance®” (Glyburide and metformin hydrochloride tablet, 1.25 mg/250 mg, 2.5 mg/500 mg, 5 mg/500 mg) is based on the teaching of the U.S. Pat. No. 6,303,146 B1 patent. By carefully selecting the particle size and limiting it to be less than 10 μm, Glucovance® has demonstrated a glyburide bioavailability equivalent to that of administering glyburide and metformin separately. Additionally, WO 01/51463 A1 discloses that having defined particle size of glyburide avoids the rapid onset of a very high concentration of glyburide in blood plasma (the “spike”), which may cause undesirable hypoglycemia to the patient.

However, both U.S. Pat. No. 6,303,146 B1 and WO 01/51463 A1 use pulverization and micronization to achieve the specific size distribution claimed in the patents. The process normally involves the use of air jet milling, which requires costly equipments (usually cost around $100,000 to $200,000), special processing areas, and adequate occupational trainings to reduce the amount of exposure to the antihyperglycemic compound used in the process. Moreover, since the compound particles are micronized by air jet with cyclone separator, accurate control of the material flow rate and segregation is necessary in order to properly separate coarse particles. In addition, the wide distribution of particle size often resulted from the pulverization and micronization processes requires repetitive micronizations, which would unavoidable cause material losses. For similar reason, strict quality control is required to achieve a reasonable reproducibility of the end product. In summary, the pulvarization and micronization processes disclosed in U.S. Pat. No. 6,303,146 B1 and WO 01/51463 A1 are costly, time consuming, and are always associated with a significant amount of material losses.

### DETAILED DESCRIPTION OF INVENTION

The present invention is directed to a process of producing a solid oral dosage comprising metformin and glyburide. The process is simple, low-cost, and integrated, and can be easily scaled for industrial manufacturing. The process comprises the steps of:

a) homogenizing glyburide in an organic solution to obtain a slurry;

b) spraying the slurry of step a) onto a pharmaceutical excipient;

c) mixing the product of step b) above with metformin granules; and

d) forming solid oral dosage of metformin and glyburide.

Alternatively, the homogenized product of step a) can be sprayed onto metformin granules directly to reduce steps b) and c) above into a single step. Further, the spraying can be performed with simultaneous drying at a temperature from 30-45° C. A fluidized bed granulator, such as Glatt®, manufactured by Glatt Air Techniques, Inc. USA, can be used in the spraying step.

In one embodiment of the invention, the organic solution in step a) above is an alcohol. Preferably, the alcohol is methanol, ethanol, or other lower alkanol(s) with 1-6 carbon atoms, their dilution(s) and mixture(s) thereof. Other organic solutions that are readily available to a person skilled in the art, such as acetone, can also be used in the current invention. In a preferred embodiment, ethanol is used for homogenizing glyburide.

The homogenizing process can be performed by using a high-speed homogenizer, such as Miccra® D-8 manufactured by ART moderne Labortechnik, Germany, which is capable to perform an homogenization at an speed of 5000 to 50,000 rpm. Preferably, homogenization is performed at a speed between 10,000 to 50,000 rpm and for a period of at least one hour. The resulted product of homogenization is a slurry with about 8-15% of glyburide by weight being in a soluble form and the remaining 85-92% of glyburide being in a solid form of fine particles, as can be determined by gravimetric estimation involving filtration and drying of the residue. To determine the size of the glyburide particles, a sieve can be used to screen the particles. In one embodiment, a sieve with a pore size of 45 μm in diameter is used. When 100% of the slurry can pass through the 45 μm sieve, the size of the glyburide particles contained in the slurry is below 45 μm in diameter. In a preferred embodiment, a sieve with a pore size of 45 μm in diameter is used. When 100% of the slurry passes through the 35 μm sieve, the glyburide particles have size values of less than 35 μm in diameter.

To increase the dissolvability of glyburide, wetting agents such as nonionic surfactants can be added into with the homogenized product before it is sprayed onto pharmaceutical excipient or metformin granules. The wetting agent that can be used in the present invention includes, but not limited to polysorbate-80 (Tween-80®), polyoxyethylene 40 hydrogenated castor oil (Cremophore® RH 40), Caprylocaproyl-macrogol-8-glyceride (Labrasol®), PEG-20 stearyl ether (Brij-78®), PEG-20 glyceryl stearate (Capmul® RTM), PEG-40 glyceryl laurate (Croda), PEG-60 corn glycerides (Crovol® M70), PEG-20 sorbitan monostearate (Tween-60®), Sucrose monostearate (Crodesta F-160®), Polaxomer 108 (Pluronic RTM).

The pharmaceutical excipient that can be employed in the present invention include, but not limited to, lactose, microcrystalline cellulose, starch, dicalcium phosphate, tricalcium phosphate, calcium carbonate, maltodextrin, colloidal silicone dioxide, and magnesium stearate, etc. Further, binders, such as polyvinylpyrrolidone (Kollidon-30) and Hydroxypropyl cellulose, can be added to the homogenized slurry of step a) above to impart binding properties to the granules.

The weight ratio of glyburide to organic solution that can be employed in the present invention is from 1/10 to 1/40, preferably from 1/20 to 1/30 by weight. The weight ratio of glyburide to wetting agent that can be employed in the present invention is from 1/1 to 1/5, preferably from 1/2 to 1/4 by weight. The weight ratio of glyburide to pharmaceutical excipient carrier that can be employed is from 1/10 to 1/80, preferably from 1/25 to 1/65 by weight.

The solid oral dosage obtained by the present invention can take the form of a tablet, a gelatin capsule, or other forms commonly known in the art. The tablets can be either unilayer or bilayer tablets, and the solid oral dosage can be film coated with hydrophilic polymers such as hydroxypropyl methylcellulose, hydroxypropyl cellulose, etc.

The solid oral dosage prepared by the current invention comprises about 8 to 15% of glyburide by weight in a soluble or molecularly dispersed form with no detectable size value. The remaining glyburide is in the form of fine dispersed particles with diameters of less than 45 μm or, preferably, less than 35 μm. The solid oral dosage of these structural characteristics is capable of producing a graded release of glyburide over a period time, i.e. an early onset of glyburide followed by a steadily increase of glyburide release. Although not to be bound by any particular theory, it appears that the initial quick onset of glyburide release is contributed by the 8-15% of glyburide by weight that is in the soluble form with no detectable size value, and the steady release is caused by the remaining 85-92% of glyburide that is in the particle form. Thus, the solid oral dosage prepared by the current invention is capable of causing an adequate and extended drug dissolvability and bioavailability without producing excessively high maximum plasma drug concentrations, i.e. the “spike”.

The resulted solid oral dosage of the current invention is also capable of producing a bioavailability and/or dissolvability of glyburide that is comparable to those of the “Glucovance®” tablet, i.e. the combination tablets with 25% undersize value not more than 6 μm, 50% undersize value not more than 7-10 μm, and 75% undersize value not more than 21 μm, as disclosed in the U.S. Pat. No. 6,303,146 B1 patent and approved by the United State Food and Drug Agency (FDA) on Jul. 31, 2000 as Glucovance® (hereinafter, the “Glucovance®” tablet).

The term “comparable” means substantially the same. Specifically, when used together with the phrase “in-vivo bioavailability”, it means that, when administered to a patient or experimental animal, the solid oral dosage form prepared by the present invention would produce a glyburide bioavailability within 80-125% of the maximum plasma concentration (Cmax) and area under plasma concentration versus time curve (AUC) of that of administering the Glucovance® tablet of the same dosage strength. When used with the phrase “in-vitro dissolvability”, the term “comparable” means that the solid oral dosage form prepared by the present invention would produce a percentage glyburide release within 80-125% of the percentage glyburide release when putting the Glucovance® tablet of the same dosage strength into the same solution.

Although metformin hydrochloride is the most commonly used metformin salt in the pharmaceutical industry, it should be understood that other forms of metformin can also be used in the current invention in lieu of metformin hydrochloride. The term “metformin,” when used in the current application, means metformin hydrochloride or other metformin salts that can be readily conceived by a person skilled in the art.

The metformin granules can be prepared by using the following procedure. Four hundred grams (400 gm) of polyvinylpyrrolidone (Kollidon-30®) is mixed with 400 gm purified water and heated to 80-90° C. temperature. One hundred grams (100 gm) of maize starch is dispersed in 150 gm of purified water and the slurry is added to above hot polyvinylpyrrolidone solution and mixed to get uniform paste. Five thousand grams (5000 gm) of metformin hydrochloride, 200 gm of sodium starch glycolate are mixed in a granulator. The polyvinylpyrrolidone-starch paste is added to the granulator such as Rapid mixer granulator (Aeromatic-Fielder, PMA) and the wet mass is granulated. The granules are dried in fluidized bed dryer and sized by using 16-mesh sieve. Alternatively the polyvinylpyrrolidone-stach paste can be sprayed on the mixture of metformin and sodium starch glyclate in a fluid bed granulator (Glatt® FBE Processor) to get the metformin granules.

In one embodiment of the invention, 50 gm of glyburide previously pulverized & milled through 0.5 mm screen is dispersed in 1180 gm of Ethanol and homogenized at 25000 rpm for 2 hours followed by passing through 35 μm mesh sieve. Then 150 gm of polyvinylpyrrolidone (Kollidon-30) and 150 gm of polysorbate-80 is dissolved in this glyburide slurry. The resulting dispersion is sprayed on the mixture of 1900 gm of lactose, 300 gm of Starch-1500® and 100 gm of sodium starch glycolate in a fluid bed granulator (Glatt® FBE Processor) with a spray rate of 30-45 gm per ml at 0.5 to 1.0 bar air pressure and 35 to 45° C. inlet air temperature. Granules thus obtained are sized through 16-mesh sieve. Alternatively the the granules colud be obtained by spraying the glyburide slurry on the mixture of lactose, Starch-1500® and sodium starch glycolate in single-pot process equipment (Zanchetta).

According to another embodiment of the current invention, 50 gm of glyburide previously pulverized & milled through 0.5 mm screen is dispersed in 1180 gm of Ethanol and homogenized at 25000 rpm for 2 hours followed by passing through 35 μm mesh sieve. Then 400 gm of polyvinylpyrrolidone (Kollidon-30) and 150 gm of polysorbate-80 is dissolved in this glyburide slurry. The resulting dispersion is sprayed on the mixture of 5000 gm of metformin hydrochloride, 1900 gm of lactose, 300 gm of Starch-1500® and 100 gm of sodium starch glycolate in a fluid bed granulator (Glatt®) with a spray rate of 30-45 gm per ml at 0.5 to 1.0 bar air pressure and 35 to 45° C. inlet air temperature. Granules thus obtained are sized through 16-mesh sieve. Alternatively the granules could be obtained by spraying the glyburide slurry on the mixture of metformin, lactose, Starch-1500® and sodium starch glycolate in single-pot process equipment (Zanchetta).

The current invention is also directed to the treatment of diabetes by administering to a patient in need thereof an effective amount of the solid oral dosage forms of the combination of glyburide and metformin prepared by the current invention.

The following examples are provided to further illustrate the present invention.

### EXAMPLE 1

The preparation of a tablet containing 500 mg of metformin and 5 mg of glyburide without homogenization and wetting agent:

Part 1—Glyburide Granules:

Fifty (50) gm of glyburide was initially pulverized and milled through 0.5 mm screen. The resulted glyburide particles were then mixed with 1900 gm of lactose, 300 gm of Starch-1500® and 100 gm of sodium starch glycolate and granulated with solution of 150 gm of polyvinylpyrrolidone (Kollidon-30) in 1180 gm of ethanol. The granules thus obtained were sized through 16-mesh sieve after drying at 45° C. temperature.

Part 2—Metformin Granules:

Four hundred (400) gm of polyvinylpyrrolidone (Kollidon-30®) was mixed with 400 gm purified water and heated to 80-90° C. temperature. One hundred (100) gm maize starch was dispersed in 150 gm of purified water and the slurry was added into the above stated hot polyvinylpyrrolidone solution and mixed to get an uniform paste. Five thousand (5000) gm of metformin hydrochloride, 200 gm of sodium starch glycolate were mixed in a granulator. The polyvinylpyrrolidone-starch paste was added to the granulator and the wet mass was granulated. The granules were dried in fluidized bed dryer and sized by using 16-mesh sieve.

Part 3-Tablet Preparation:

Metformin granules (Part 2) were mixed with glyburide granules (part 1), 70 gm of colloidal silicone dioxide and 30 gm of magnesium stearate. The granule mixture was tabletted by using a suitable tablet press. The tablets obtained were then coated with a 0.850% hydroxypropyl cellulose (Methocel® E15) in a tablet coating machine.

### EXAMPLE 2

The preparation of a tablet containing 500 mg of metformin and mg of glyburide with homogenization but without wetting agent (i.e. polysorbate-80):

Part 1—Glyburide Granules:

Fifty (50) gm of glyburide was initially pulverized and milled through 0.5 mm screen. The resulted glyburide particles were then dispersed in 1180 gm of ethanol and homogenized at 25000 rpm for 2 hours to obtain a slurry. Subsequently, the slurry was screened by passing through a 35 μm mesh sieve and 150 gm of polyvinylpyrrolidone (Kollidon-30) was added into the glyburide slurry. The resulting dispersion was sprayed on the mixture of 1900 gm of lactose, 300 gm of Starch-1500® and 100 gm of sodium starch glycolate in a fluid bed granulator (Glatt®) with a spray rate of 30-45 gm per ml at 0.5 to 1.0 bar air pressure and 35 to 45° C. inlet air temperature. Granules thus obtained were sized through 16-mesh sieve.

Part 2—Metformin Granules:

Four hundred (400) gm of polyvinylpyrrolidone (Kollidon-30®) was mixed with 400 gm purified water and heated to a temperature of 80-90° C. One hundred (100) gm of maize starch was dispersed in 150 gm of purified water and the slurry was added to above hot polyvinylpyrrolidone solution and mixed to get uniform paste. Five thousand (5000) gm of metformin hydrochloride and 200 gm of sodium starch glycolate were mixed in a granulator. The polyvinylpyrrolidone-starch paste was added to the granulator such as Rapid mixer granulator (Aeromatic-Fielder, PMA) and the wet mass was granulated. The granules were dried in fluidized bed dryer and sized by using 16-mesh sieve.

Part 3-Tablet Preparation:

The glyburide granules (part 1) and the metformin granules (Part 2) were mixed as well as 70 gm of colloidal silicone dioxide and 30 gm of magnesium stearate. The granule mixture was tabletted by using a suitable tablet press. The tablets obtained were then coated with a 0.85% hydroxypropyl cellulose (Methocel® E15) in a tablet coating machine.

### EXAMPLE 3

The preparation of a tablet containing 500 mg of metformin and 5 mg of glyburide with homogenization and wetting agent (polysorbate-80):

Part 1—Glyburide Granules:

Fifty (50) gm of glyburide was initially pulverized and milled through 0.5 mm screen. The resulted glyburide particles were then dispersed in 1180 gm of ethanol and homogenized at 25000 rpm for 2 hours to obtain a slurry. Subsequently, the slurry was screened by passing through a 35 μm mesh sieve, and 150 gm of polyvinylpyrrolidone (Kollidon-30) and 150 gm of polysorbate-80 were added into the glyburide slurry. The resulting dispersion was sprayed on the mixture of 1900 gm of lactose, 300 gm of Starch-1500® and 100 gm of sodium starch glycolate in a fluid bed granulator (Glatt®) with a spray rate of 30-45 gm per ml at 0.5 to 1.0 bar air pressure and 35 to 45° C. inlet air temperature. Granules thus obtained were sized through 16-mesh sieve.

Part 2—Metformin Granules:

Four hundred (400) gm of polyvinylpyrrolidone (Kollidon-30®) was mixed with 400 gm purified water and heated to a temperature of 80-90° C. One hundred (100) gm of maize starch was dispersed in 150 gm of purified water and the slurry was added to above hot polyvinylpyrrolidone solution and mixed to get uniform paste. Five thousand (5000) gm of metformin hydrochloride and 200 gm of sodium starch glycolate were mixed in a granulator. The polyvinylpyrrolidone-starch paste Was added to the granulator and the wet mass was granulated. The granules were dried in fluidized bed dryer and sized by using 16-mesh sieve.

Part 3—Tablet Preparation:

The glyburide granules (part 1) and the metformin granules (Part 2) were mixed with 70 gm of colloidal silicone dioxide and 30 gm of magnesium stearate. The granule mixture was tabletted by using a suitable tablet press. The tablets obtained were then coated with a 0.85% hydroxypropyl cellulose (Methocel® E15) in a tablet coating machine.

### EXAMPLE 4

The preparation of a tablet containing 500 mg of metformin and mg of glyburide with homogenization and wetting agent (Polyoxyethylene 40 hydrogenated castor oil (Cremophore® RH 40):

Part 1—Glyburide Granules:

Fifty (50) gm of glyburide was initially pulverized and milled through 0.5 mm screen. The resulted glyburide particles were then dispersed in 1180 gm of ethanol and homogenized at 25000 rpm for 2 hours to obtain a slurry. Subsequently, the slurry was screened by passing through a 35 μm mesh sieve, and 150 gm of polyvinylpyrrolidone (Kollidon-30) and 150 gm of Polyoxyethylene 40 hydrogenated castor oil (Cremophore® RH 40) were added into the glyburide slurry. The resulting dispersion was sprayed on the mixture of 1900 gm of lactose, 300 gm of Starch-1500® and 100 gm of sodium starch glycolate in a fluid bed granulator (Glatt®) with a spray rate of 30-45 gm per ml at 0.5 to 1.0 bar air pressure and 35 to 45° C. inlet air temperature. Granules thus obtained were sized through 16-mesh sieve.

Part 2—Metformin Granules:

Four hundred (400) gm of polyvinylpyrrolidone (Kollidon-30®) was mixed with 400 gm purified water and heated to a temperature of 80-90° C. One hundred (100) gm of maize starch was dispersed in 150 gm of purified water and the slurry was added to above hot polyvinylpyrrolidone solution and mixed to get uniform paste. Five thousand (5000) gm of metformin hydrochloride and 200 gm of sodium starch glycolate were mixed in a granulator. The polyvinylpyrrolidone-starch paste was added to the granulator and the wet mass was granulated. The granules were dried in fluidized bed dryer and sized by using 16-mesh sieve.

Part 3—Tablet Preparation:

The glyburide granules (part 1) and the metformin granules (Part 2) were mixed with 70 gm of colloidal silicone dioxide and 30 gm of magnesium stearate. The granule mixture was tabletted by using a suitable tablet press. The tablets obtained were then coated with a 0.85% hydroxypropyl cellulose (Methocel® E15) in a tablet coating machine.

### EXAMPLE 5

The preparation of a tablet containing 500 mg of metformin and 5 mg of glyburide with homogenization and wetting agent (polysorbate-80):

Part 1—Glyburide Granules:

Fifty (50) gm of glyburide was initially pulverized and milled through 0.5 mm screen. The resulted glyburide particles were then dispersed in 1180 gm of ethanol and homogenized at 25000 rpm for 2 hours to obtain a slurry. Subsequently, the slurry was screened by passing through a 35 μm mesh sieve, and 400 gm of polyvinylpyrrolidone (Kollidon-30) and 150 gm of polysorbate-80 were added into the glyburide slurry. The resulting dispersion was sprayed on the mixture of 5000 gm of metformin hydrochloride, 1900 gm of lactose, 300 gm of Starch-1500® and 100 gm of sodium starch glycolate in a fluid bed granulator (Glatt®) with a spray rate of 30-45 gm per ml at 0.5 to 1.0 bar air pressure and 35 to 45° C. inlet air temperature. Granules thus obtained were sized through 16-mesh sieve.

Part 2—Tablet Preparation:

The granules obtained in part 1 were mixed with 70 gm of colloidal silicone dioxide and 30 gm of magnesium stearate. The granule mixture was tabletted by using a suitable tablet press. The tablets obtained were then coated with a 0.85% hydroxypropyl cellulose (Methocel® E15) in a tablet coating machine.

### EXAMPLE 6

The preparation of a tablet containing 500 mg of metformin and 2.5 mg of glyburide with homogenization and wetting agent (polysorbate-80):

Part 1—Glyburide Granules:

Twenty-five (25) gm of glyburide was initially pulverized and milled through 0.5 mm screen. The resulted glyburide particles were then dispersed in 590 gm of ethanol and homogenized at 25000 rpm for 2 hours to obtain a slurry. Subsequently, the slurry was screened by passing through a 35 μm mesh sieve, and 75 gm of polyvinylpyrrolidone (Kollidon-30) and 75 gm of polysorbate-80 were added into the glyburide slurry. The resulting dispersion was sprayed on the mixture of 950 gm of lactose, 150 gm of Starch-1500® and 50 gm of sodium starch glycolate in a fluid bed granulator (Glatt®) with a spray rate of 30-45 gm per ml at 0.5 to 1.0 bar air pressure and 35 to 45° C. inlet air temperature. Granules thus obtained were sized through 16-mesh sieve.

Part 2—Metformin Granules:

Four hundred (400) gm of polyvinylpyrrolidone (Kollidon-30®) was mixed with 400 gm purified water and heated to a temperature of 80-90° C. One hundred (100) gm of maize starch was dispersed in 150 gm of purified water and the slurry was added to above hot polyvinylpyrrolidone solution and mixed to get uniform paste. Five thousand (5000) gm of metformin hydrochloride and 200 gm of sodium starch glycolate were mixed in a granulator. The polyvinylpyrrolidone-starch paste Was added to the granulator and the wet mass was granulated. The granules were dried in fluidized bed dryer and sized by using 16-mesh sieve.

Part 3—Tablet Preparation:

The glyburide granules (part 1) and the metformin granules (Part 2) were mixed with 70 gm of colloidal silicone dioxide and 30 gm of magnesium stearate. The granule mixture was tabletted by using a suitable tablet press. The tablets obtained were then coated with a 0.85% hydroxypropyl cellulose (Methocel® E15) in a tablet coating machine.

### EXAMPLE 7

The preparation of a tablet containing 250 mg of metformin and 1.25 mg of glyburide with homogenization and wetting agent (polysorbate-80):

Part 1—Glyburide Granules:

Twelve and a half (12.5) gm of glyburide was initially pulverized and milled through 0.5 mm screen. The resulted glyburide particles were then dispersed in 295 gm of ethanol and homogenized at 25000 rpm for 2 hours to obtain a slurry. Subsequently, the slurry was screened by passing through a 35 μm mesh sieve, and 37.5 gm of polyvinylpyrrolidone (Kollidon-30) and 37.5 gm of polysorbate-80 were added into the glyburide slurry. The resulting dispersion was sprayed on the mixture of 475 gm of lactose, 75 gm of Starch-1500® and 25 gm of sodium starch glycolate in a fluid bed granulator (Glatt®) with a spray rate of 30-45 gm per ml at 0.5 to 1.0 bar air pressure and 35 to 45° C. inlet air temperature. Granules thus obtained were sized through 16-mesh sieve.

Part 2—Metformin Granules:

Two hundred (200) gm of polyvinylpyrrolidone (Kollidon-30®) was mixed with 200 gm purified water and heated to a temperature of 80-90° C. Fifty (50) gm of maize starch was dispersed in 75 gm of purified water and the slurry was added to above hot polyvinylpyrrolidone solution and mixed to get uniform paste. Two thousand and five hundred (2500) gm of metformin hydrochloride and 100 gm of sodium starch glycolate were mixed in a granulator. The polyvinylpyrrolidone-starch paste Was added to the granulator and the wet mass was granulated. The granules were dried in fluidized bed dryer and sized by using 16-mesh sieve.

Part 3—Tablet Preparation:

The glyburide granules (part 1) and the metformin granules (Part 2) were mixed with 35 gm of colloidal silicone dioxide and 15 gm of magnesium stearate. The granule mixture was tabletted by using a suitable tablet press. The tablets obtained were then coated with a 0.85% hydroxypropyl cellulose (Methocel® E15) in a tablet coating machine.

### EXAMPLE 8

The preparation of a capsule containing 250 mg of metformin and 1.25 mg of glyburide with homogenization and wetting agent (polysorbate-80):

Part 1—Glyburide Granules:

Twelve and a half (12.5) gm of glyburide was initially pulverized and milled through 0.5 mm screen. The resulted glyburide particles were then dispersed in 295 gm of ethanol and homogenized at 25000 rpm for 2 hours to obtain a slurry. Subsequently, the slurry was screened by passing through a 35 μm mesh sieve, and 37.5 gm of polyvinylpyrrolidone (Kollidon-30) and 37.5 gm of polysorbate-80 were added into the glyburide slurry. The resulting dispersion was sprayed on the mixture of 475 gm of lactose, 75 gm of Starch-1500® and 25 gm of sodium starch glycolate in a fluid bed granulator (Glatt®) with a spray rate of 30-45 gm per ml at 0.5 to 1.0 bar air pressure and 35 to 45° C. inlet air temperature. Granules thus obtained were sized through 16-mesh sieve.

Part 2—Metformin Granules:

Two hundred (200) gm of polyvinylpyrrolidone (Kollidon-30®) was mixed with 200 gm purified water and heated to a temperature of 80-90° C. Fifty (50) gm of maize starch was dispersed in 75 gm of purified water and the slurry was added to above hot polyvinylpyrrolidone solution and mixed to get uniform paste. Two thousand and five hundred (2500) gm of metformin hydrochloride and 100 gm of sodium starch glycolate were mixed in a granulator. The polyvinylpyrrolidone-starch paste Was added to the granulator and the wet mass was granulated. The granules were dried in fluidized bed dryer and sized by using 16-mesh sieve.

Part 3—Tablet Preparation:

The glyburide granules (part 1) and the metformin granules (Part 2) were mixed with 35 gm of colloidal silicone dioxide and 15 gm of magnesium stearate. The granule mixture is filled in size 00 hard gelatin capsule.

### EXAMPLE 9

### In-Vitro Dissolvability Study

The in-vitro dissolvability of the tablets prepared by the above examples and a Glucovance™ tablet marketed by Bristol-Myers Squibb Company USA were tested in 900 ml phosphate buffer pH 7.5 USP by using a USP Dissolution apparatus II at a speed of 100 rpm.

| Tablets | Method of | | | |
|---|---|---|---|---|
| (Metformin/ | Preparation | | | |
| Glyburide | 1) Homogenization | % glyburide release after | | |
| w/w) | 2) Wetting agent | 30 min | 45 min | 60 min |
| Example 1 | 1) No | <50 | <50 | <50 |
| (500/5) | 2) No | | | |
| Example 2 | 1) Yes | 61.9-63.78 | 72.3-74.9 | 75.4-81.8 |
| (500/5) | 2) No | | | |
| Example 3 | 1) Yes | 76.7-83.25 | 87.1-91.36 | 86.3-96.2 |
| (500/5) | 2) Yes | | | |
| | (polysorbate-80) | | | |
| Example 4 | 1) Yes | 87.59-94.23 | 95.86-101.29 | 98.52-100.1 |
| (500/5) | 2) Yes | | | |
| | (polyoxyethylene | | | |
| | 40 hydrogenated | | | |
| | castor oil) | | | |
| Example 5 | 1) Yes | 77.5-84.3 | 86.5-92.6 | 86.6-94.5 |
| (500/5) | 2) Yes | | | |
| | [Sprayed Directly | | | |
| | onto Metformin | | | |
| | Granules] | | | |
| Example 6 | 1) Yes | 83.2-90.8 | 90.35-97.2 | 92.4-100.7 |
| (500/2.5) | 2) Yes | | | |
| Example 7 | 1) Yes | 83.1-92.4 | 92.1-97.05 | 92.7-100.3 |
| (250/1.25) | 2) Yes | | | |
| Example 8 | 1) Yes | 81.52-87.4 | 85.4-91.06 | 89.9-94.65 |
| (250/1.25) | 2) Yes | | | |
| | [Gelatin Capsule] | | | |
| Glucovance ® | 1) No | 74.1-84.41 | 79.82-85.2 | 84.6-88.8 |
| Tablet | 2) No | | | |
| (500/5) | | | | |
| Glucovance ® | 1) No | 76.4-82.4 | 81.7-84.9 | 83.8-86.2 |
| Tablet | 2) No | | | |
| (500/2.5) | | | | |
| Glucovance ® | 1) No | 79.3-80.7 | 83.6-85.09 | 86.25-87.9 |
| Tablet | 2) No | | | |
| (250/1.25) | | | | |

Example 1 showed a tablet prepared by traditional method with no homogenization of glyburide and no addition of wetting agent. Examples 2-8 were directed to metformin/glyburide combination tablets prepared by the current invention. No wetting agent was added in Example 2, while Example 4 showed a process wherein the glyburide slurry was directly sprayed onto metformin granules.

The in-vitro dissolvability data clearly showed that the solid oral dosage prepared by homogenizing glyburide in organic solution (Examples 2-8) produced significant higher glyburide in-vitro dissolvability when compared with the tablet prepared by traditional method (Example 1). When wetting agents such as nonionic surfactants polysorbate-80 or polyoxyethylene 40 hydrogenated castor oil was added into the glyburide slurry, as in Examples 3-8, a further improvement in in-vitro dissolvability of glyburide was observed.

In addition, the solid oral dosages prepared by the process of the present invention (Examples 2-8) produced a graded release of glyburide over a period of 60 minutes: there was an initial quick in-vitro release of glyburide which entailed an early onset of glyburide followed by steadily increasing glyburide release. It appeared that the initial quick onset of glyburide release was contributed by the 8-15% of glyburide that was in the soluble form and the stead release was contributed by the 85-92% of glyburide that was in the dispersed form. The consequence is: the solid oral dosage prepared by the current invention ensured an adequate and extended drug dissolvability and bioavailability without producing excessively high maximum plasma drug concentrations, i.e. the “spike”.

Further, the solid oral dosages prepared by the process of the present invention (Examples 2-8) produced an in-vitro dissolvability that was comparable to that of the Glucovance® tablet. Stated in another way, the solid oral dosage form prepared by the present invention would produce a percentage glyburide release within 80-125% of the percentage glyburide release when putting the Glucovance® tablet of the same dosage strength into the same solution.

### EXAMPLE 9

### In-Vivo Bioavailability Study

An in-vivo bioavailability study was conducted in human volunteers, encompasses:

1. The compositions of Example 2 above and the currently marketed product Glucovance™ (500/5) were administered orally to human volunteers using a crossover, randomized design under fasting conditions and the resulting bioavailability of glyburide was measured and compared.

The mean pharmacokinetic parameters such as Maximum plasma concentration (Cmax), Time to reach maximum plasma concentration (Tmax), Area under the drug plasma concentration versus time curve (AUC0-t & AUC0-∞) were noted for glyburide and the values observed were as follow.

TABLE 2
Comparison of the in-vivo bioavailabilities of the tablets
of Example 2 and the Glucovance ® tablets.

| Parameters | Glucovance ® (500/5) | Tablets of Example 2 |
|---|---|---|
| Cₘₐₓ (ng/ml) | 187.71 ± 72.2 | 158.5 ± 80.48 |
| AUC₀₋ₜ (ng/ml*hour) | 1108.56 ± 642.65 | 731.1 ± 441.75 |
| AUC_{0-∞} (ng/ml*hour) | 1197 ± 642.65 | 838.29 ± 441.75 |

TABLE 3
90% Confidence Interval (C.I.) for Logtransformed data for Glibenclamide
for Example 2 with respect to Glucovance ®.

| | | US FDA |
|---|---|---|
| | | Acceptance criteria |
| Parameters | 90% C.I. | for 90% C.I. ** |
| Cmax (ng/ml) | 89.73-103.42 | 80-125 |
| AUC (0-t) (ng/ml*hr) | 88.94-99.76 | 80-125 |
| AUC (0-∞) (ng/ml*hr) | 90.78-99.98 | 80-125 |

(90% C.I. meets the US FDA Acceptance criteria for log-transformed data to document bio-equivalence)
** = Guidance for Industry: Food effect Bioavailability and fed bioequivalence studies. U.S. FDA, CDER, December 2002 guidelines.

2. The compositions of Example 3 above and the currently marketed product Glucovance® (500/5) were administered orally to human volunteers using a crossover, randomized design under fasting conditions and the resulting bioavailability of glyburide was measured and compared.

The mean pharmacokinetic parameters such as Maximum plasma concentration (Cmax), Time to reach maximum plasma concentration (Tmax), Area under the drug plasma concentration versus time curve (AUC0-t & AUC0-∞) were noted for glyburide and the values observed were as follow.

TABLE 4
Comparison of the in-vivo bioavailabilities of the tablets
of Example 3 and the Glucovance ® tablets.

| Parameters | Glucovance ® (500/5) | Tablets of Example 3 |
|---|---|---|
| Cₘₐₓ (ng/ml) | 164.226 ± 26.114 | 147.94 ± 38.333 |
| AUC₀₋ₜ (ng/ml*hour) | 653.359 ± 242.494 | 595.6371 ± 302.663 |
| AUC_{0-∞} (ng/ml*hour) | 682.124 ± 242.494 | 629.439 ± 302.663 |

3. The composition of Example 3 above is administered orally to human volunteers. Separate and simultaneous administration of the currently marketed products Glucophage® Tablet containing 500 mg of Metformin hydrochloride manufactured by Bristol-Myers Suibb and Diabeta® Tablet containing 5 mg of Glyburide manufactured by Aventis, USA, were also conducted on human volunteer.

A randomized, single dose, two treatment, two period, open label, cross-over study design with a washout period was used to evaluate the relative bioavailability of the composition of Example 3 with separate and simultaneous administration of Metformin hydrochloride (Glucophage® Tablet-500 mg) and Glyburide (Diabeta®-5 mg tablet) in healthy, adult, male subjects under fasting conditions.

The mean pharmacokinetic parameters such as Maximum plasma concentration (Cmax), Time to reach maximum plasma concentration (Tmax), Area under the drug plasma concentration versus time curve (AUC0-t & AUC0-∞) were noted for Glyburide and the values observed were as follow.

TABLE 6
Comparison of the in-vivo bioavailabilities of the tablets
of Example 3 and the in-vivo bioavailabilities of separate
and simultaneous administration of Metformin and Glyburide.

| | Diabeta ® 5 mg + | |
|---|---|---|
| Parameters | Glucophage ® 500 mg | Tablets of Example 3 |
| Cₘₐₓ (ng/ml) | 116.997 ± 15.780 | 128.154 ± 22.316 |
| AUC₀₋ₜ (ng/ml*hour) | 527.516 ± 228.114 | 469.194 ± 111.905 |
| AUC_{0-∞} (ng/ml*hour) | 581.948 ± 228.115 | 520.883 ± 111.9 |

TABLE 7
90% Confidence Interval (C.I.) for Logtransformed (In) data for
Glibenclamide for Example 3 with respect to combined administration
of Diabeta ® 5 mg and Glucophage ® 500 mg.

| | | US FDA |
|---|---|---|
| | | Acceptance criteria |
| Parameters | 90% C.I. | for 90% C.I. ** |
| In Cmax (ng/ml) | 100.62-106.59 | 80-125 |
| In AUC (0-t) | 93.50-101.55 | 80-125 |
| (ng/ml*hr) | | |
| In AUC (0-∞) | 93.97-101.15 | 80-125 |
| (ng/ml*hr) | | |

(90% C.I. meets the US FDA Acceptance criteria for log-transformed data to document bio-equivalence)
** = Guidance for Industry: Food effect Bioavailability and fed bioequivalence studies. U.S. FDA, CDER, December 2002 guidelines.

The data showed:

1. The compositions of Example 2 and 3 produced no significant difference with respect to the in-vivo bioavailability of glyburide.

2. The composition of Example 3 produced comparable glyburide bioavailability of that of Glucovance®.

3. The composition of Example 3 produced comparable glyburide bioavailability when comparing with that of the separate and simultaneous administration of metformin and glyburide.

In summary, the solid oral dosage form of metformin and glyburide that is generated by the present process is capable of producing a glyburide bioavailability and/or dissolvability comparable to that of administering the Glucovance® tablet of the same strength. The solid oral dosage form of metformin and glyburide generated by the present process is also capable of producing a glyburide bioavailability that is comparable to that of separately and simultaneously administering the tablets of glyburide and metformin of the same strenth.

We claim:

1. A process of producing a solid oral dosage comprising metformin and glyburide, the process comprising the steps of:
a) homogenizing glyburide in an organic solution to obtain a slurry;
b) spraying the slurry onto a pharmaceutical excipient;
c) mixing the product of step b) above with metformin granules; and
d) forming solid oral dosage of metformin and glyburide.

2. The process of claim 1, wherein the slurry of step a) comprises about 8-15% of glyburide by weight in a soluble form with no detectable size value and the remaining glyburide in the form of particles with diameters of less than 45 μm.

3. The process of claim 1, wherein the organic solution is methanol, ethanol, or other lower alkanol(s) with 1-6 carbon atoms, and mixture(s) thereof.

4. The process of claim 3, wherein the organic solution is ethanol.

5. The process of claim 1, further comprising the step of adding a wetting agent to the product of step a).

6. The process of claim 5, wherein the wetting agent is selected from the group consisting of polysorbate-80 and polyoxyethylene 40 hydrogenated castor oil.

7. The process of claim 1, wherein the spraying as in step b) is performed with simultaneous drying.

8. The process of claim 7, wherein the simultaneous drying is conducted at a temperature between 30-45° C.

9. The process of claim 1, further comprising the step of screening the product of step a) by a sieve with a pore size of 45 μm in diameter, wherein 100% of the product of step a) passes through the sieve.

10. The process of claim 9 wherein the pore size is 35 μm in diameter.

11. The process of claim 1, wherein the weight ratio of metformin to glyburide is 50/1 to 250/1.

12. The process of claim 1, wherein the homogenization is performed at 5,000 to 50,000 rpm.

13. The process of claim 1, wherein the homogenization is performed for a period of not less than 1 hour.

14. The process of claim 1, wherein the weight ratio of glyburide to organic solution is 1/10 to 1/40.

15. The process of claim 1, wherein the spraying is performed on a fluidized bed granulator.

16. The process of claim 1, wherein the solid oral dosage produced in step d) is in the form of tablet.

17. The process of claim 1, wherein the solid oral dosage produced in step d) is in the form of gelatin capsule.

18. A process of making a solid oral dosage form of the combination of metformin and glyburide, comprising:
a) homogenizing glyburide in an organic solution to obtain a slurry;
b) spraying the slurry onto metformin granules; and
c) forming solid oral dosage of metformin and glyburide.

19. A solid oral dosage comprising metformin and glyburide, wherein about 8-15% of glyburide by weight is in the soluble form with no detectable size value and the remaining glyburide is in the form of particles with diameters of less than 45 μm.

20. The solid oral dosage form of claim 19, further comprising a wetting agent.

21. The solid oral dosage form of claim 20, wherein the wetting agent is selected from the group consisting of polysorbate-80 and polyoxyethylene 40 hydrogenated castor oil.

22. A method of treating non-insulin dependent diabetes or hyperglycemia, comprising administering to a subject in need thereof an effective amount of the solid oral dosage of claim 19.

23. Use of the solid oral dosage of claim 19, in the manufacture of a medicament for treating non-insulin dependent diabetes or hyperglycemia.
